(19) United States
(12) Patent Application Publication
(10) Pub. No.: US 2005/0215995 A1
(43) Pub. Date: Sep. 29, 2005

(54) Electromagnetic field surgical device and method

(75) Inventor: Isamu Nakahira, Tokyo (JP)

Correspondence Address: KARL R CANNON, PO BOX 1909, SANDY, UT, 84091, US

(73) Assignee: Japan Medical Dynamic Marketing, Inc.

(21) Appl. No.: 11/129,720

(22) Filed: May 13, 2005

Related U.S. Application Data
(63) Continuation of application No. 11/005,554, filed on Dec. 6, 2004, now abandoned.
Application No. 11/005,554 is a continuation of application No. 10/866,109, filed on Jun. 10, 2004, now abandoned.
Application No. 10/866,109 is a continuation of application No. 10/703,760, filed on Nov. 7, 2003, now abandoned.
Application No. 10/703,760 is a continuation-in-part of application No. 10/407,854, filed on Apr. 4, 2003, now abandoned.
Application No. 10/407,854 is a continuation of application No. 10/262,553, filed on Sep. 30, 2002, now abandoned.
Application No. 10/262,553 is a continuation of application No. 10/112,584, filed on Mar. 29, 2002, now abandoned.
(60) Provisional application No. 60/280,010, filed on Mar. 30, 2001.

(51) Int. Cl. A61B 18/14

(52) U.S. Cl. 606/45; 606/49

(57) ABSTRACT

An electromagnetic field surgical device for cutting and vaporizing tissue, and coagulating fluids. The device may include a surgical tool or probe having an electrode forming a tip. An electromagnetic field may be radiated from the tip of the surgical tool. The surgical tool may be placed in close proximity to the tissue to be treated to form a gap between the electrode and the tissue. An arc of current may be discharged from the tip of the electrode through the tissue to cut and vaporize the tissue. The transfer of energy from the electrode to the tissue may be optimized by moving the electrode. An output unit in the device may include a high frequency isolation transformer, and low frequency cut-off circuit to protect the patient from low frequency energy. Current may be switched through different circuits having fixed impedances to perform different tissue treatments.

### CROSS-REFERENCE TO RELATED APPLICATIONS

This application is a continuation of co-pending U.S. patent application Ser. No. 11/005,554, filed Dec. 6, 2004, entitled “ELECTROMAGNETIC FIELD SURGICAL DEVICE AND METHOD,” which is a continuation of U.S. patent application Ser. No. 10/866,109, filed Jun. 10, 2004, entitled “ELECTROMAGNETIC FIELD SURGICAL DEVICE AND METHOD,” which is a continuation of U.S. patent application Ser. No. 10/703,760, filed Nov. 7, 2003, entitled “ELECTROMAGNETIC FIELD SURGICAL DEVICE AND METHOD,” which is a continuation-in-part of U.S. patent application Ser. No. 10/407,854, filed Apr. 4, 2003, entitled “ELECTROMAGNETIC FIELD SURGICAL DEVICE AND METHOD,” which is a continuation of U.S. patent application Ser. No. 10/262,553, filed Sep. 30, 2002, entitled “ELECTROMAGNETIC FIELD SURGICAL DEVICE AND METHOD,” which is a continuation of U.S. patent application Ser. No. 10/112,584, filed Mar. 29, 2002, entitled “ELECTROMAGNETIC FIELD SURGICAL DEVICE AND METHOD,” which claims the benefit of U.S. Provisional Application No. 60/280,010, filed Mar. 30, 2001, entitled “ELECTROMAGNETIC FIELD SURGICAL DEVICE AND METHOD,” which applications are hereby incorporated by reference herein in their entireties, including but not limited to those portions that specifically appear hereinafter, the incorporation by reference being made with the following exception: In the event that any portion of the above-referenced applications is inconsistent with this application, this application supercedes said above-referenced applications.

### STATEMENT REGARDING FEDERALLY SPONSORED RESEARCH OR DEVELOPMENT

Not Applicable.

### BACKGROUND

1. The Field of the Invention

The present disclosure relates generally to a device and method for using an electromagnetic field for surgical procedures, and more particularly, but not necessarily entirely, to a surgical instrument producing an electromagnetic field for cutting, vaporizing tissue, and coagulating blood vessels.

2. Description of Related Art

Surgical instruments are known in the art for use in cutting, cauterizing and vaporizing along a thin incision as well as coagulating fluids so that surgical procedures may be performed without bleeding. For example, mono-polar electrocautery systems have been in use for some time in coagulating vessels and for cutting tissue. In the prior electrocautery systems, high frequency electric current is passed from a cautery probe through the tissue to a grounding pad. Heat is generated in the tissue at the site of contact of the probe tip to the tissue by the flow of energy through the electrical resistance of the tissue in the preferred path between the probe tip contact site and the grounding pad. In such devices, the energy is continuous sinusoidal or amplitude modulated. The heat generated by the cautery of the prior mono-polar electrocautery systems is not uniform since the heating of the tissue is greater in the preferred path of current of lower resistance. For this reason, as the current flows from the point of contact of the probe to the surrounding tissue, heating also tends to spread beyond the contact point of the probe to the surrounding tissue thereby causing damage to the surrounding tissue.

Some of the problems associated with the prior mono-polar electrocautery systems were overcome by the bi-polar cautery system which typically uses forceps. Current flows from one tip of the forceps to the other tip of the forceps without the spread of current to the surrounding tissues. Both the mono-polar electrocautery and the bi-polar cautery system can cut tissue and coagulate vessels but cannot vaporize tissue.

A lesion generator known as a radio frequency lesion generator is known in the art and works on the same principles as the mono-polar cautery system except that a lower level of current is used and the current is of the continuous sinusoidal type. This current type results in more uniform tissue destruction. However, such a system is used exclusively for creating lesions.

A system using a radio frequency surgical tool was developed to overcome some of the problems of the prior art systems. The radio frequency surgical tool is capable of cutting and vaporizing tissue and coagulating vessels without the spread of heat to the surrounding tissue. A high frequency (13.56 or 27.0 MHZ) current is passed through an amplifier, a matching network and a solenoid coil to generate an electromagnetic field. This in turn induces eddy currents in the tissue. Touching the tissue with a probe which is AC-coupled to a return circuit draws the eddy currents out of the tissue at the contact point of the probe producing intense heat which can cut and vaporize tissue as well as coagulate vessels. One disadvantage of this system is that the proximity of the coil to the operative field causes inconvenience to the surgeon. A further disadvantage of this device is that the coagulating ability of the device is not as efficient as desired. Another disadvantage of the device is that it requires a grounding component.

An electroconvergent cautery system was developed as a surgical tool for coagulating blood vessels and cutting and vaporizing tissue. In an electroconvergent cautery system, electrical current is passed through either a surgical probe or forceps. The current is generated by a radio frequency power generator which produces an alternating current of 13.56 or 27.0 MHZ. An impedance matching device is utilized to match the impedance of the probe or the active blade of the forceps with the radio frequency power generator. A loading tuning coil serves as an auto transformer which minimizes the mismatch of impedance of the probe or the active blade of the forceps with the radio-frequency generator upon touching the tip of the probe or the active blade of the forceps to the tissue. This causes the current to converge to the tip and results in high current density at the tip of the probe or the active blade of the forceps. Furthermore, the loading and tuning coil instantaneously causes the current at the probe tip to capacitatively couple with the return circuit, drawing back the current into the return circuit. The high current density at the sharp tip of the probe or the active blade of the forceps produces intense localized heating which is capable of coagulating vessels and cutting and vaporizing tissue. As the current is instantaneously drawn back into the return circuit, the heat is restricted to the contact point. When vessels are held between the two tips of the forceps some energy is dissipated into the inactive blade resulting in diffuse heating which improves its coagulating property.

Despite the advantages of the electroconvergent cautery system, the electroconvergent cautery system requires various components such as a loading and tuning coil, and an impedance matching device, which increase the complexity of the device. Furthermore, the electroconvergent cautery system does not isolate the patient from dangerous low frequency energy or provide separate circuits with fixed impedance for cutting or coagulating, and a switch to control the flow of current through the circuits. Also, the electroconvergent cautery system does not utilize the impedance of specialized connecting cables to achieve a fixed optimal efficiency setting.

In view of the foregoing state of the art, it would be an advancement in the art to provide an electromagnetic field surgical device which can cut and vaporize tissue, and can coagulate fluids without spreading heat to the surrounding tissue. It would be a further advancement in the art to provide an electromagnetic field surgical device which eliminates the need for a loading and tuning coil, and a grounding component, and which can be easily manipulated. It would also be an advancement in the art to provide an electromagnetic field surgical device which can achieve optimal energy transfer to tissue by moving the device with respect to the tissue, and which allows for pre-set power/impedance which can be selectively controlled by diverting current through specialized circuits with a switch. It would be a further advancement in the art to provide an electromagnetic field surgical device which isolates the patients from dangerous low frequency energy, and which utilizes the impedance of connecting cables to achieve optimal efficiency.

The prior art is thus characterized by several disadvantages that are addressed by the present disclosure. The present disclosure minimizes, and in some aspects eliminates, the above-mentioned failures, and other problems, by utilizing the methods and structural features described herein.

### BRIEF DESCRIPTION OF THE DRAWINGS

The above and other, features and advantages of the disclosure will become apparent from a consideration of the subsequent detailed description presented in connection with the accompanying drawings in which:

FIG. 1 is a schematic view of an electromagnetic field surgical device made in accordance with the principles of the present disclosure;

FIG. 2a is a side view of a divided cable and mono-polar probe;

FIG. 2b is a side view of a divided cable and a bi-polar probe;

FIG. 3 is a side view of an exemplary embodiment of a mono-polar probe arranged to allow a clear line of sight during use;

FIG. 4 is a schematic view of a transmission path of a radio frequency energy and an electromagnetic wave energy when a mono-polar probe is use-d in accordance with the principles of the present disclosure;

FIG. 5 is a schematic view of a transmission path of a radio frequency energy and an electromagnetic wave energy when a bi-polar probe is used in accordance with the principles of the present disclosure;

FIG. 6 is a schematic view of a transmission path of a current and a control signal from a power supply of the electromagnetic field surgical device to the probe; and

FIG. 7 is a schematic view of the components of the output unit connected to the cable and surgical tool of the present disclosure.

### DETAILED DESCRIPTION

For the purposes of promoting an understanding of the principles in accordance with the disclosure, reference will now be made to the embodiments illustrated in the drawings and specific language will be used to describe the same. It will nevertheless be understood that no limitation of the scope of the disclosure is thereby intended. Any alterations and further modifications of the inventive features illustrated herein, and any additional applications of the principles of the disclosure as illustrated herein, which would normally occur to one skilled in the relevant art and having possession of this disclosure, are to be considered within the scope of the disclosure claimed.

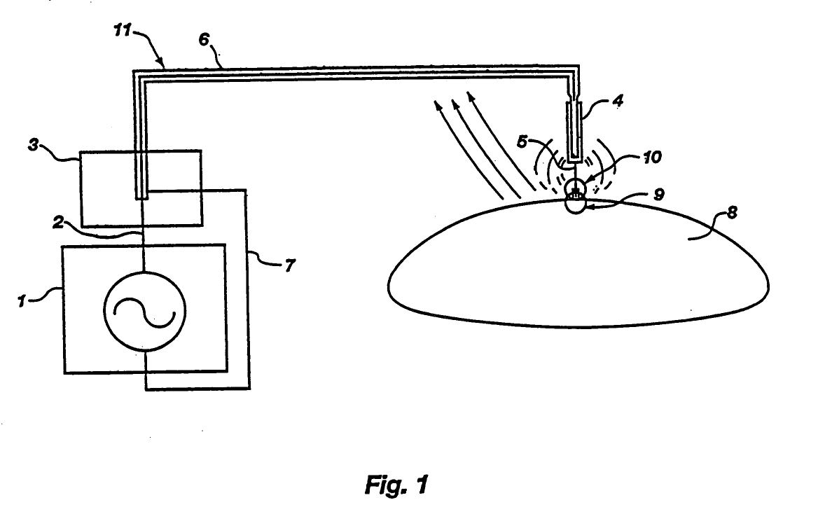

Referring now to FIG. 1, a schematic view is shown of an electromagnetic field surgical device made in accordance with the principles of the present disclosure. The electromagnetic field surgical device may include a radio frequency power source 1, also sometimes referred to as a radio frequency generator. The power source 1 may be capable of generating a high radio frequency energy, such as current of at least 8 MHZ to 60 MHZ, or higher for example. A cable 11 may connect the power source 1 with a surgical tool or probe 4. The cable 11 may have a core wire 2 and a shielded wire 6, that coaxially encloses the core wire 2 through insulating material. The core wire 2 may be connected to a conductor of the power source 1, and the shielded wire 6 may be connected to another conductor of the power source 1 through a lead wire 7. The other end of the core wire 2 may be connected to the surgical tool 4. The shielded wire 6 may enclose the core wire 2 to a position near the tip of the surgical tool 4. Thus, when a current is transmitted from the power source 1 to the surgical tool 4, the shielded wire 6 effectively captures the electromagnetic wave radiated from the core wire 2. As a result, the energy radiated as an electromagnetic wave from the core wire 2 may be prevented from dissipating into the air.

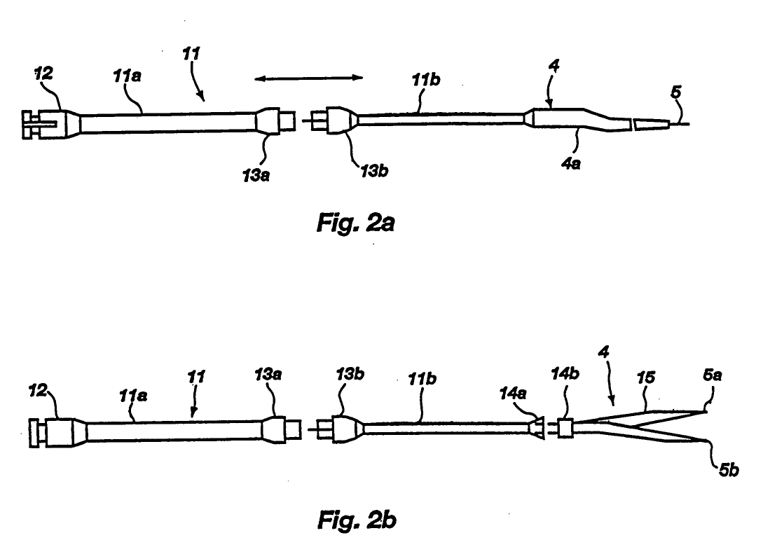

The surgical tool 4 may take the form of various mono-polar or bi-polar configurations as illustrated in FIGS. 2a and 2b. For example, the surgical tool 4, illustrated in FIG. 2a may comprise a mono-polar probe having an electrode 5 which may be arranged to be replaceably attached to an active output terminal inside the surgical tool 4. The word “active” as used herein refers to an element that is a source of electrical energy, or capable of converting or amplifying voltages or currents. “Passive” as used herein refers to elements exhibiting no gain or contributing no energy.

As shown in FIG. 2b, the surgical tool 4 may comprise a bi-polar probe having blades 15 with electrode tips 5a, 5b. Electrode tip 5a may be connected to the active output terminal inside the surgical tool 4, whereas the tip 5b may be connected to the passive output terminal inside the surgical tool 4. It will be appreciated by those skilled in the art that surgical tools 4 of various different configurations may be attached to the cable 11 through connectors 14a, 14b.

Also, as shown in FIG. 2, the cable 11 may be divided into a plurality of sections having different diameters. The larger diameter cable portions 11a allow the device to operate more efficiently due to the decreased resistance provided by the larger-diameter. The smaller diameter cable portions 11b allow the device to be more flexible which improves the ability to manipulate the device. By connecting a small diameter cable 11b to a larger diameter cable 11a through a connector 13a, 13b, the device is able achieve benefits of both efficiency and flexibility.

A tip of the surgical tool 4 may include the electrode 5 which is supplied with a radio frequency through the core wire 2 from the power source 1. The electrode 5 may radiate a strong electromagnetic wave from its tip. The electrode 5 may be positioned in a region in close proximity to the tissue 8 that is to be surgically treated to form a gap, shown generally at 10 in FIG. 1, between the tissue 8 and the electrode 5. The tissue 8 may then be exposed to the electromagnetic field, and an arc may be discharged between the electrode 5 and the tissue 8 within the gap 10. With the tissue 8 serving as a ground, the arc current may flow into a local region of the tissue, shown generally at 9, to locally generate a Joule heat, and thereby vaporize the tissue 8 to cut and/or cauterize the tissue 8.

Unlike prior art devices, the electrode 5 of the present disclosure may utilize the gap 10 to provide optimal cutting and vaporizing of the tissue 8. The electrode 5 may be placed as close to the tissue 8 as possible without actually touching the tissue 8. In the event the tissue 8 is inadvertently contacted by the electrode 5, the efficiency of the electromagnetic field surgical device for cutting and vaporizing may be reduced. However, when the electromagnetic field surgical device is used for coagulating fluids, optimal efficiency of the device may be achieved when the electrode 5 contacts the tissue 8. This allows the surgeon to press the electrode against the tissue 8 to pinch blood vessels for example, to enhance the coagulation process. The electromagnetic field surgical device may be placed in different operating modes to achieve optimal cutting or coagulating as discussed more fully below.

The radio frequency energy may be directly supplied to the electrode 5 through the cable 11 from the power source 1. Therefore, an electromagnetic coil such as used in prior art devices is not needed. The surgical device can therefore be made smaller and lighter so that it is easier to handle and operate. Furthermore, the elimination of the electromagnetic coil facilitates operating the surgical device without obstructing the view of the surgeon.

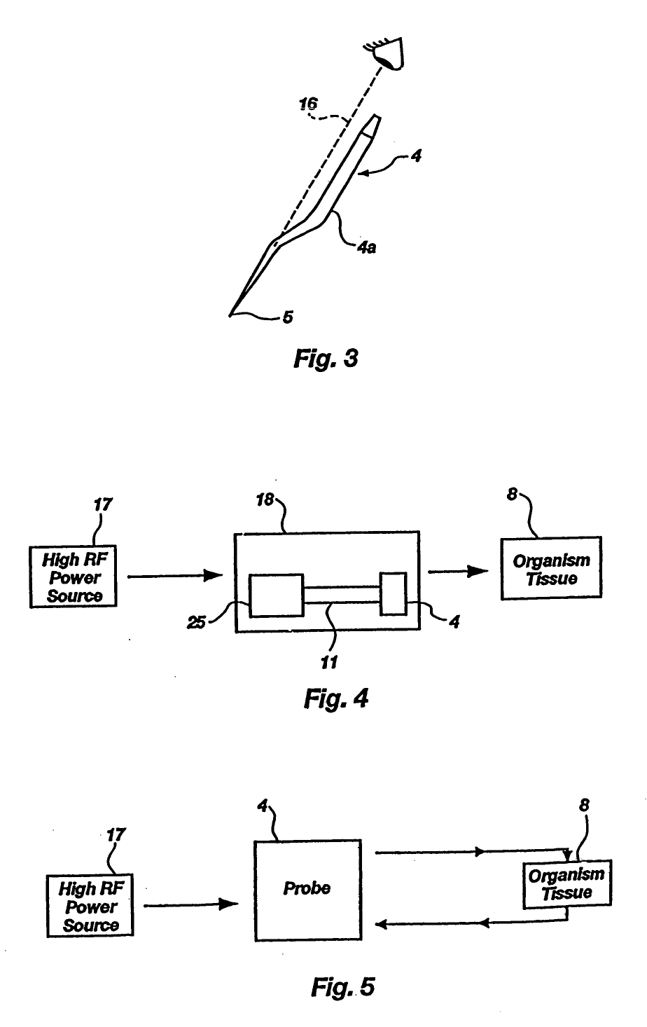

As shown in FIG. 3, the view of the surgeon may be further enhanced by forming the surgical tool 4 in a bent or offset configuration. The surgical tool 4 may be arranged to be offset from the electrode 5 and the line of sight 16 of the surgeon. This allows the surgeon to grip the surgical tool 4 without obstructing the line of sight 16 with the surgical tool 4 or the surgeon's hand.

FIG. 4 illustrates a transmission path of a radio frequency energy and an electromagnetic wave energy when a mono-polar type probe is used in the electromagnetic field surgical device. A high radio frequency power source 17 may generate a high band radio frequency which may be supplied to an energy converter 18. The energy converter 18 may include an output unit 25, a cable 11, and a surgical tool 4. The energy converter 18 may provide a strong electromagnetic wave which radiates from a tip of the electrode 5 in the surgical tool 4. When the tip of the electrode 5 is placed in close proximity to a local region 9 of a tissue 8, the tissue 8 may be exposed to an electromagnetic field. An arc may be discharged in the gap 10 between the tip of the electrode 5 and the tissue 8, or current may flow into the local region 9 of the tissue 8, the tissue 8 serving as a ground, to locally generate a Joule heat. The arc discharge and the Joule heat allow for treatment of the tissue, such as to cut and/or cauterize the tissue and coagulate fluids.

In contrast, FIG. 5 illustrates a transmission path of a radio frequency energy and an electromagnetic wave energy in use with a bi-polar probe. The power source 17 may generate a high band radio frequency which may be supplied to the electrode 5a of the surgical tool 4. The electrode 5a and a facing electrode 5b form a bi-polar electrode. The electrode 5a may radiate a strong electromagnetic field from a tip thereof. Similar to the mono-polar probe discussed above, when the tip of The electrode 5a is positioned in a region in close proximity to the tissue 8 that is to be surgically treated, a gap 10 may be formed between the tissue 8 and the electrode 5a. The tissue 8 may then be exposed to the electromagnetic field, and an arc may be discharged between the electrode 5a and the tissue 8 within the gap 10. However, at the same time, an arc current may flow into the tip of the facing electrode 5b through the local region of tissue 9 to create a local Joule heat in the local region of the tissue 9. As a result, cutting, vaporizing and cauterizing of the tissue 8 may be accomplished. In the bi-polar probe, the electrode 5a may be connected to an active output terminal, and the facing electrode 5b may be connected to a passive output terminal, or the facing electrode 5b may be maintained in the open state without being connected to the passive terminal.

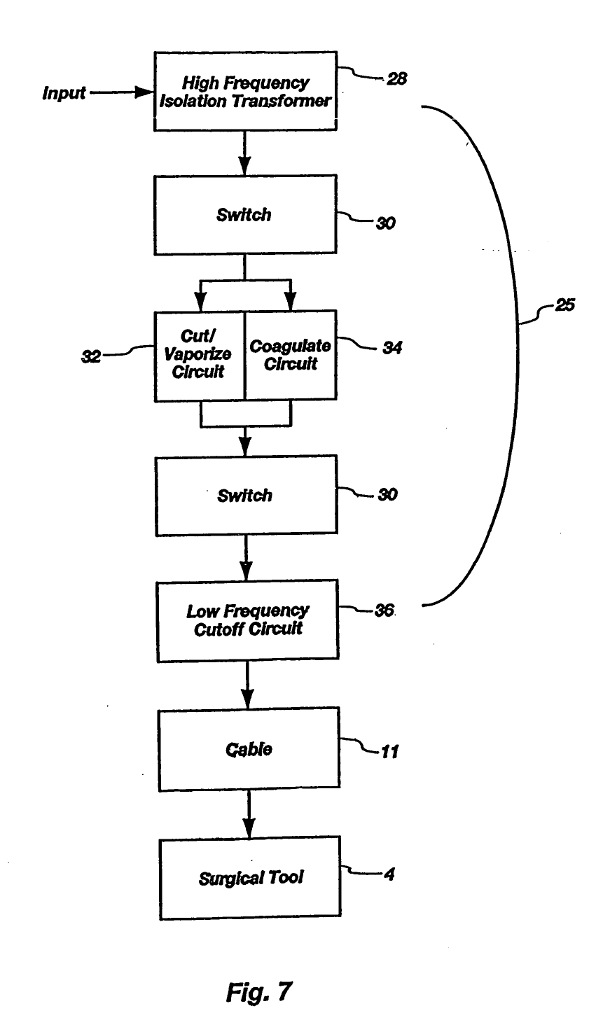

In both the mono-polar and bi-polar configurations, the electrode 5 may be connected to the passive output terminal through an impedance circuit, shown as items 32 and 34 in FIG. 7 and discussed more fully below. The impedance circuit may include at least one capacitor and at least one inductor. The radio frequency characteristics of the radio frequency energy flowing through the electrode 5 may be varied in accordance with the construction of the impedance circuit between the electrode 5 and the passive output terminal. Thus, the optimum radio frequency characteristics may be selected in accordance with the requirements for the treatment to the tissue 8.

The surgeon may also match the impedance by adjusting the distance between the electrode 5 and the tissue 8 for optimal energy transfer across the gap 10 and into the tissue 8. Cutting of the tissue 8 occurs optimally when the electrode 5 is located as close as possible to the tissue 8 without touching the tissue 8. As the tissue 8 is cut, the distance between the electrode 5 and the tissue 8 increases due to the vaporizing of the tissue 8. The surgeon may move the electrode 5 closer to the tissue 8 to optimize the energy transfer across the gap 10 and continue to cut the tissue 8. The optimal impedance and energy transfer for coagulating occurs when the electrode 5 contacts the tissue 8, thus the surgeon may merely touch the tissue 8 with the electrode 5 to achieve optimal coagulation efficiency.

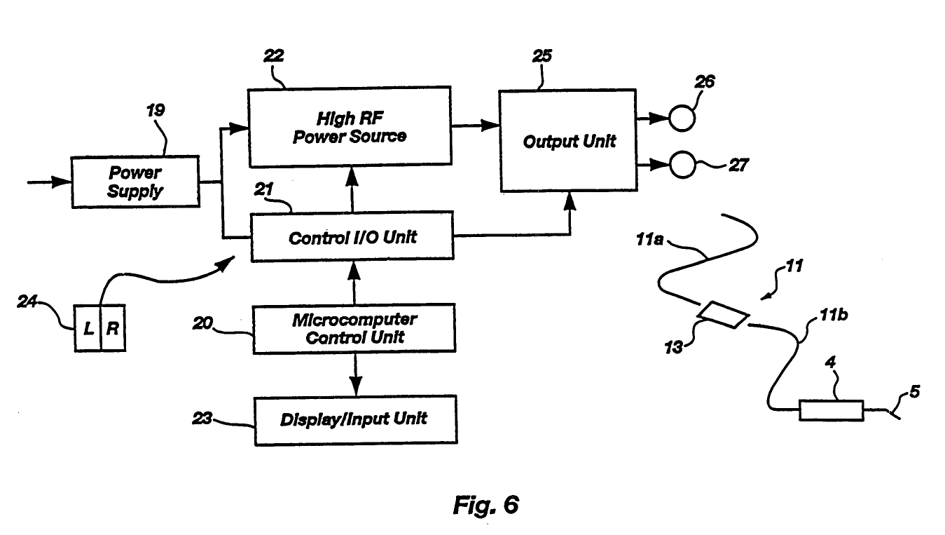

FIG. 6 illustrates one example of a transmission path of a current and a control signal from a power supply of the electromagnetic field surgical device using a radio frequency surgical tool 4. A power supply 19, of a variety known in the art, may be provided to supply a current. The current may be converted into a radio frequency of at least a high band radio frequency, for example, a frequency covering 8 MHZ to 60 MHZ, or higher by a high radio frequency power source 22. The radio frequency energy generated by the high radio frequency power source 22 may be transmitted to an output unit 25 having a mono-polar output unit 26 and a bi-polar output unit 27.

A microcomputer control unit 20 may execute an output control of the high radio frequency power source 22 and a matching control of the output unit 25 through a control input/output (I/O) unit 21, and render a display/input unit 23 to display necessary items relating to the output state of the high radio frequency power source 22 and the matching operation state of the output unit 25, and the like. A foot switch or pedal switch 24 may be used to control the I/O unit 21. Pressing the pedal switch 24 may operate to connect or disconnect the output of the output unit 25, or change the mode of the device to cut or coagulate.

The surgical tool or probe 4 may be connected to the output unit 25 through the cable 11 including the divided cable 11a of a larger diameter, the relay connector 13, and the divided cable 11b of a smaller diameter. In the case of a surgical tool 4 having a mono-polar type electrode 5, the cable 11 may be connected to the mono-polar output unit 26 of the output unit 25; whereas in the case of a bi-polar type surgical tool 4, the cable may be connected to the bi-polar output unit 27 of the output unit 25.

The output form of a radio frequency energy can be reshaped to enhance the effect of a treatment to an organism tissue 8. For example, the power level, amplitude and frequency of the current may be adjusted, modulated or pulsed to achieve a desired effect such as improved cutting, coagulating, or preventing burnt deposits from forming on the tip of the electrode 5.

FIG. 7 shows a schematic diagram of the components of the output unit, indicated generally at 25. Current generated by the high radio frequency power source 22 may enter the output unit 25 as input. The output unit 25 may include a high frequency isolation transformer 28 or other filtering mechanism to separate out low frequency energy. The high frequency isolation transformer 28 is one example of a high frequency isolation transformer means for separating out low frequency energy. This enhances patient safety since low frequency energy can be harmful to the patient. As referred to herein, “low frequency” may include radio frequencies in the range from about 30 to 300 kilohertz, or lower, for example. The high frequency isolation transformer 28 maybe of any variety of high frequency isolation transformers known in the art for separating high frequency energy from low frequency energy. This isolates the output unit 25 from low frequency energy present at the high radio frequency power source 22.

The output unit 25 preferably includes two circuits, a cutting and/or vaporizing circuit 32 to provide optimal efficiency for cutting and/or vaporizing tissue, and a separate coagulation circuit 34 for providing optimal efficiency in coagulating fluids. The cutting and/or vaporizing circuit 32 and the coagulating circuit 34 may include a combination of one or more capacitors and one or more inductive coils to establish a preset impedance which may be optimized for the specific function of the circuit. In addition, the circuits 32 and 34 may include one or more variable components that allow adjustment of the impedance of circuits 32 and 34 by a user of the surgical tool.

The output unit 25 may also include at least one switch mechanism 30 for controlling the flow of current through the cutting and/or vaporizing circuit 32 and the coagulating circuit 34. The switch 30 is one example of switch means for controlling the flow of current in the circuits 32, 34. The switch 30 may be formed in any manner known in the art for directing or regulating current flow, such as by means of relays, solid state silicon chips, or transistors for example. The output unit 25 may include two switches 30 at opposite ends of the cutting and/or vaporizing circuit 32 and the coagulating circuit 34, which operate together to control the flow of current in the circuits. However, it will be appreciated that the switch 30 may be located at either end of the cutting and/or vaporizing circuit 32 and the coagulating circuit 34, as well as at both ends to control the flow of current through the circuits. In the preferred implementation, switch 30 performs a mutually exclusive switching function wherein the coagulate circuit 34 is disconnected when the cut/vaporize circuit 32 is coupled between the high radio frequency power source and the surgical tool, and wherein the cut/vaporize circuit 32 is disconnected when the coagulate circuit 34 is coupled between the high radio frequency power source and the surgical tool. It will also be appreciated that any number of circuits may be used within the scope of the present disclosure to establish optimal working characteristics for an intended use of the electromagnetic field surgical device.

The cut and/or vaporize circuit 32 may provide an impedance which causes the energy from the electromagnetic field emitted from the surgical tool 4 to focus so that cutting and vaporizing of the tissue can be accomplished with optimal efficiency. In contrast, the coagulation circuit 34 may provide an impedance which causes the electromagnetic field emitted from the surgical tool 4 to disperse so that coagulation of fluids occurs efficiently. The impedance of the gap 10 may be considered when establishing the impedance of the cut and/or vaporize circuit 32 such that an optimal energy output exists when a gap 10 is present. The coagulation circuit 34 may provide optimal energy output when the surgical tool comes into contact with the tissue 8. The surgical tool 4 may therefore be used in the coagulation mode to apply pressure to the tissue 8 and pinch blood vessels to enhance the coagulation effects of the electromagnetic field surgical device.

The switch 30 may direct the current through a selected circuit to accomplish the desired treatment of the tissue. The switch 30 is controlled by the control I/O unit 21 (FIG. 6), which may be activated by depressing the pedal 24 to cause the electromagnetic field surgical device to operate using either the cut/vaporize circuit 32 or the coagulation circuit 34 to either cut tissue 8 or coagulate fluids. The characteristics of the electromagnetic field can be further modified by modulating or pulsing the current through one of the circuits to accomplish a combination of cutting and coagulating. For example, a blend mode which accomplishes cutting of the tissue 8 and coagulating of fluid may be accomplished by modulating the frequency and pulsing the current through the cut and/or vaporization circuit 32. For example, in a cut or coagulate mode, the frequency may be 13.56 MHZ, and 100 percent of the cycle, continuous sinusoidal current, may be used as output from the output unit 25. Whereas in a blend mode, the frequency may be modulated to 13.56 kHz and the current may be pulsed, or turned on for a portion of a cycle and turned off for a portion of the cycle. An exemplary blend mode may have ninety percent on time and ten percent off time. However, it will be appreciated that other modulated frequencies and on/off percentages can be used within the scope of the present disclosure to accomplish the desired blend of cutting and coagulation.

The output unit 25 may also include a low frequency cut-off circuit 36 to remove low frequency energy from the current. The low frequency cut-off circuit 36 may also be referred to as a high pass filter or a means for removing low frequency energy from the current. Those skilled in the art will appreciate that components of various different configurations may be used to remove low frequency energy from the current within the scope of the present disclosure. This provides additional safety to patients using the electromagnetic field surgical device since some low frequency energy may pass through the high frequency isolation transformer 28, and low frequency energy may be generated in the circuitry after the current passes through the high frequency isolation transformer 28.

Output from the output unit 25 may pass through the cable 11 to the surgical tool 4. The cable 11 may have characteristics that are important to the circuitry in the electromagnetic field surgical device. For example, the length, diameter and material type of the cable 11 may all contribute to the impedance of the cables 11. The impedance values of the cutting and/or vaporizing circuit 32 and the coagulating circuit 34 may be established with a particular impedance value of the cable 11. Therefore, if the impedance characteristics of the cable 11 are changed, corresponding changes in the cutting and/or vaporizing circuit 32 and the coagulating circuit 34 may be required to achieve optimal efficiency in the electromagnetic field surgical device. The cable 11 may be of a variety known in the art having resistance values of between 50 and 70 ohms for example. The cable 11 may have a length in the range of between 3.5 to 4.0 meters. The larger diameter portion 11a may have a length in a range of between 2.0 to 3.0 meters, whereas the smaller diameter portion lib may have a length in a range of 0.5 to 1.5 meters. However, it will be appreciated by those skilled in the art that the cable 11 may have various other lengths and impedance characteristics within the scope of the present disclosure.

It will be appreciated that the structure and apparatus disclosed herein is merely one example of a means for removing low frequency energy from the current, and it should be appreciated that any structure, apparatus or system for removing low frequency energy from the current which performs functions the same as, or equivalent to, those disclosed herein are intended to fall within the scope of a means for removing low frequency energy from the current, including those structures, apparatus or systems for removing low frequency energy from the current which are presently known, or which may become available in the future. Anything which functions the same as, or equivalently to, a means for removing low frequency energy from the current falls within the scope of this element.

It will be appreciated that the structure and apparatus disclosed herein is merely one example of a high frequency isolation transformer means for separating out low frequency energy, and it should be appreciated that any structure, apparatus or system for separating out low frequency energy which performs functions the same as, or equivalent to those disclosed herein are intended to fall within the scope of a high frequency isolation transformer means for separating out low frequency energy, including those structures, apparatus or systems for separating out low frequency energy which are presently known, or which may become available in the future. Anything which functions the same as, or equivalently to, high frequency isolation transformer means for separating out low frequency energy falls within the scope of this element.

It will be appreciated that the structure and apparatus disclosed herein is merely one example of a switch means for controlling the flow of current, and it should be appreciated that any structure, apparatus or system for controlling the flow of current which performs functions the same as, or equivalent to, those disclosed herein are intended to fall within the scope of a switch means for controlling the flow of current, including those structures, apparatus or systems for controlling the flow of current which are presently known, or which may become available in the future. Anything which functions the same as, or equivalently to, a switch means for controlling the flow of current falls within the scope of this element.

In accordance with the features and combinations described above, a method for surgically treating tissue 8 in a patient may include the steps of
(a) selecting one of a first impedance and a second impedance to couple between a high radio frequency power source and a surgical tool, the first impedance providing a first mode of operation of the surgical tool and the second impedance providing a second mode of operation of the surgical tool;
(b) radiating an electromagnetic field from a tip of said surgical tool by coupling the high radio frequency power source through one of the first impedance and second impedance to the surgical tool; and
(c) placing the tip of said surgical tool in close proximity to the tissue that is to be surgically treated.

It will be appreciated that the device may include a power source which may generate an energy having a preselected frequency. The power source may be connected to a surgical tool or probe through a cable having a core wire and a coaxial shielded wire. The impedance of the cable may be selected to achieve optimal energy transfer. The disclosure may also include an output box having separate circuits, one to accomplish cutting of tissue, and the other to accomplish coagulating of fluids. The flow of current through the circuits may be controlled by one or more switches. The output unit may be isolated from dangerous low frequency energy by a high frequency isolation transformer. An additional low frequency cut-off circuit may be included in the output unit to further protect the patient from dangerous low frequency energy. The disclosure may include an electrode having a tip. The tip of the electrode may be placed in close proximity to the tissue to be treated to form a gap between the tissue and the tip of the electrode for use in cutting the tissue, or the tip of the electrode may contact the tissue for optimal efficiency when coagulating fluids. An electromagnetic field may be radiated from the tip of the electrode and an arc of current may be discharged from the tip through the gap and into the tissue to cut and vaporize the tissue. The flow of current through the tissue creates Joule heat which further serves to cut the tissue and coagulate blood. The distance between the tip of the electrode and the tissue may be adjusted to optimize the energy transfer between the electrode and the tissue.

In view of the foregoing, it will be appreciated that the present disclosure provides an electromagnetic field surgical device which can cut and vaporize tissue, and can coagulate fluids without spreading heat to the surrounding tissue. The present disclosure also provides an electromagnetic field surgical device which may eliminate the need for a loading and tuning coil, and a grounding component, and which can be easily manipulated. The present disclosure also provides an electromagnetic field surgical device which can achieve optimal energy transfer to tissue by moving the device with respect to the tissue, and which can allow for pre-set power/impedance which can be selectively controlled by diverting current through specialized circuits with a switch. The present disclosure also provides an electromagnetic field surgical device which may isolate the patients from dangerous low frequency energy, and which may utilize the impedance of connecting cables to achieve optimal efficiency.

In the foregoing Detailed Description, various features of the present disclosure are grouped together in a single embodiment for the purpose of streamlining the disclosure. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment. Thus, the following claims are hereby incorporated into this Detailed Description of the Disclosure by this reference, with each claim standing on its own as a separate embodiment of the present disclosure.

It is to be understood that the above-described arrangements are only illustrative of the application of the principles of the present disclosure. Numerous modifications and alternative arrangements may be devised by those skilled in the art without departing from the spirit and scope of the present disclosure and the appended claims are intended to cover such modifications and arrangements. Thus, while the present disclosure has been shown in the drawings and fully described above with particularity and detail in connection with what is presently deemed to be the most practical and preferred embodiments of the disclosure, it will be apparent to those of ordinary skill in the art that numerous modifications, including, but not limited to, variations in size, materials, shape, form, function and manner of operation, assembly and use may be made without departing from the principles and concepts set forth herein.

1-23. (canceled)

24. A method for surgically treating tissue in a patient comprising the steps of:
(a) selecting one of a first impedance and a second impedance to couple between a high radio frequency power source and a surgical tool, the first impedance providing a first mode of-operation of the surgical tool and the second impedance providing a second mode of operation of the surgical tool,
(b) radiating an electromagnetic field from a tip of the surgical tool by coupling the high radio frequency power source through one of the first impedance and second impedance to the surgical tool; and
(c) placing the tip of the surgical tool in close proximity to the tissue that is to be surgically treated.

25. The method of claim 24 wherein the surgical tool comprises a mono-polar probe.

26. The method of claim 24 wherein the surgical tool comprises a bi-polar probe.

27. The method of claim 24 wherein step (c) comprises the step of providing a gap between the surgical tool and the tissue.

28. The method of claim 27 further comprising discharging an arc of current from the tip of the surgical tool through the gap and into the tissue to thereby treat the tissue.

29. The method of claim 24 wherein step (c) comprises the step of contacting the tissue with the tip of the surgical tool.

30. The method of claim 24 wherein step (c) vaporizes and cuts the tissue and coagulates body fluids.

31. The method of claim 24 wherein the selection of one of the first impedance and second impedance in step (a) is performed with a foot pedal.

32. The method of claim 24 further comprising the step of providing a display of operational parameters of the tissue treatment process.

33. A method for surgically treating tissue in a patient comprising the steps of:
(a) selecting a surgical tool for treating the tissue;
(b) generating alternating current of a pre-selected frequency with a radio frequency generator;
(c) connecting the surgical tool to the radio frequency generator through a cable and one of a first circuit and a second circuit, each of the first circuit and the second circuit and the cable having predefined impedance characteristics;
(d) radiating an electromagnetic field from the surgical tool; and
(e) placing the surgical tool in close proximity to the tissue to thereby treat the tissue.

34. The method of claim 33 further comprising the step of switching between the first circuit and the second circuit to alter the treatment to the tissue.

35-45. (canceled)

46. A method for generating an electromagnetic field at a tip of a surgical tool, the method comprising the steps of:
(a) generating a high radio frequency signal;
(b) passing the high radio frequency signal to an input of a high frequency isolation mechanism;
(c) passing an output of the high frequency isolation mechanism to an input of a low frequency cutoff circuit; and
(d) routing an output of the low frequency cutoff circuit to the tip of the surgical tool.

47. The method of claim 46 wherein step (c) comprises the steps of:
(c1) passing the output of the high frequency isolation mechanism to an input of an impedance circuit that provides first and second modes of operation for the surgical tool; and
(c2) passing an output of the impedance circuit to the low frequency cutoff circuit.

48. The method of claim 47 wherein the first mode of operation comprises a cutting mode.

49. The method of claim 47 wherein the second mode of operation comprises a coagulating mode.

50. The method of claim 46 wherein the surgical tool comprises a mono-polar probe.

51. The method of claim 46 wherein the surgical tool comprises a bi-polar probe.

52. A method for surgically treating tissue in a patient comprising the steps of:
(a) selecting a surgical tool for treating the tissue;
(b) generating alternating current of a pre-selected frequency with a radio frequency generator;
(c) passing the alternating current from an output of the radio frequency generator through a high frequency isolation mechanism;
(d) passing an output of the high frequency isolation mechanism through an impedance circuit that provides a cutting mode of operating and a coagulating mode of operation for the surgical tool;
(e) passing an output of the impedance circuit through a low-frequency cutoff circuit that attenuates frequencies below a threshold frequency value;
(f) passing an output of the low-frequency cutoff circuit to the surgical tool, thereby causing the surgical tool to radiate an electromagnetic field; and
(g) placing the surgical tool in close proximity to the tissue to thereby treat the tissue.

53. The method of claim 52 further comprising the step of switching between the cutting mode of operation and the coagulating mode of operation.

54. An electromagnetic field device for surgically treating tissue of a patient, the device comprising:
a high radio frequency power source;
a surgical tool connected to the high radio frequency power source for emitting an electromagnetic field to treat the tissue;
an output unit, the output unit comprising a high frequency isolation transformer to filter out low frequency energy such that the surgical tool can be placed in close proximity to the tissue to be treated without transferring low frequency energy to the tissue;
wherein the output unit further comprises a first circuit having a first impedance and a second circuit having a second impedance, wherein the first impedance and second impedance are adjustable by a user of the surgical tool;
wherein the first impedance is fixed to provide optimal cutting of the tissue;
wherein the second impedance is fixed to provide optimal coagulation of fluids in the tissue;
wherein the output unit further comprises a switch mechanism to control the flow of current in the first and second circuits, the switch mechanism performing a mutually exclusive switching function wherein the second circuit is disconnected when the first circuit is coupled between the high radio frequency power source and the tip of the surgical tool, and wherein the first circuit is disconnected when the second circuit is coupled between the high radio frequency power source and the tip of the surgical tool;
wherein the output unit further comprises a low frequency cut-off circuit to remove low frequency energy from the electromagnetic field device to prevent the patient from being exposed to low-frequency energy;
wherein the electromagnetic field device further comprises a cable connected to the output unit and the surgical tool;
wherein the cable has a first section having a first diameter, and a second section having a second diameter, and wherein the first diameter is larger than the second diameter, wherein the cable has a fixed impedance that modifies the first impedance when the first circuit is couple between the high radio frequency power source and the tip of the surgical tool, and that modifies the second impedance when the second circuit is coupled between the high radio frequency power source and the tip of the surgical tool;
wherein the first section of the cable has a length in a range of between 2 and 3 meters;
wherein the second section of the cable has a length in a range of between 0.5 and 1.5 meters; and
wherein the surgical tool includes an offset portion to allow grasping the surgical tool in a location out of a line of sight with the tip.
